(19) United States
(12) Patent Application Publication
(10) Pub. No.: US 2017/0360687 A1
(43) Pub. Date: Dec. 21, 2017

(54) SUBSTRATE-BASED PRODUCT, ITS METHOD OF PREPARATION AND COSMETIC USES THEREOF

(71) Applicant: L'OREAL, Paris (FR)

(72) Inventors: Gaurav AGARWAL, Kawazaki-Shi Kanagawa Ken (JP); Vishal JAVIA, Rajkot (IN)

(73) Assignee: L'OREAL, Paris (FR)

(21) Appl. No.: 15/538,856

(22) PCT Filed: Dec. 23, 2015
(86) PCT No.: PCT/EP2015/081222

§ 371 Date: Jun. 22, 2017

(30) Foreign Application Priority Data
Dec. 23, 2014 (IN) 3875/DEL/2014

(51) Int. Cl. A61K 8/81; A61K 8/67; A45D 44/00; A61K 8/02; A61Q 19/00

(52) U.S. Cl. CPC A61K 8/8176; A61K 8/0212; A61Q 19/00; A45D 44/002; A61K 8/676; A61K 2800/54; A61K 2800/28

(57) ABSTRACT

The present invention concerns a substrate-based product comprising a water-insoluble substrate, at least one chemical exfoliator, and at least one skin binding agent, its method of preparation and its cosmetic uses.

The present invention concerns a substrate-based product, in particular a substrate-based cosmetic mask, its method of preparation and its cosmetic uses.

Human skin is composed of three layers: hypodermis, dermis and epidermis. The uppermost layer, epidermis, is formed of keratinocytes. Human skin is regenerated naturally as keratinocytes are displaced towards the superficial layer of epidermis, the stratum corneum. It takes the keratinocytes about two weeks to move from the stratum basal to the top of the stratum granulosum and additional four weeks to cross the stratum corneum. Thus, the entire epidermis is replaced by new cell growth over a period of about forty-eight days.

The artificial resurfacing of the skin is a well-known method to improve its smoothness and/or to improve the aspect of fine lines, wrinkles and various types of changes in pigmentation, such as spots, flecks, freckles and chloasma or heat burns. Chemical peeling is one of the most common procedures employed for artificial resurfacing and is performed thanks to the use of external compositions comprising chemical exfoliators.

However, these conventional external compositions for the chemical peeling drip at the time of their use and thickeners are needed to obtain compositions which can be applied on the skin zone to be peeled. These compositions, composed of large amounts of thickeners, are often difficult to wash away at the time of removal.

There is thus a need for improved peeling systems. In particular, there is a need for novel peeling systems which are easy to apply and easy to remove by the users. There is also a need for efficient and fast peeling systems.

One of the aims of the present invention is to provide a substrate-based product, in particular useful for skin peeling.

One of the aims of the present invention is to provide an improved and efficient skin peeling product, especially a peeling mask, more particularly a face mask.

Another aim of the present invention is to provide a kit comprising a substrate-based product and a composition, easy to use for the consumers.

Another aim of the invention is to provide a method of preparation for the substrate-based product.

One aim of the invention is also to provide a cosmetic care method, using the substrate-based product.

The invention thus relates to a substrate-based product comprising:
a water-insoluble substrate,
at least one chemical exfoliator, and
at least one skin binding agent.

The invention also concerns a method for preparing a substrate-based product according to the invention, comprising a step of application of a composition comprising at least one chemical exfoliator and at least one skin binding agent on a water-insoluble substrate, said composition being here-below called “preparation composition”.

The invention further relates to a method of cosmetic care comprising the following steps:
a) wetting with a physiologically acceptable aqueous composition at least one zone of the substrate-based product according to the invention,
b) applying the activated substrate-based product on at least one zone of skin,
c) drying the activated substrate-based product, and
d) removing the activated substrate-based product from the skin.

The invention also relates to a kit comprising the substrate-based product according to the invention, and a physiologically acceptable aqueous composition, optionally comprising at least one skin care agent.

In one embodiment, the preparation composition and the aqueous composition are two different compositions.

The procedure of controlled destruction of the uppermost layers of the epidermis, in particular of the stratum corneum for resurfacing the skin is known as chemical peeling. Conventional peeling compositions comprising chemical exfoliators and thickeners are known. The use of thickeners such as xanthan gum, polyacrylamides or hydroxylethylcellulose, with hydroxyacids has been mentioned for example in patent applications JP 5 (1993)-139947, JP 10 (1998)-218753 or JP 8 (1996)-53322. Patent application EP 2 450 029 A1 mentions that such conventional preparations comprising hydroxyacids and thickeners have bad viscosity stability.

Surprisingly, the inventors have found that a substrate-based product according to the invention is convenient to use and gives improved efficacy for skin smoothening. The combination of the chemical effect obtained by a chemical exfoliator and of a mechanical effect provided by the skin binding agent during the removal of the substrate-based product from the skin, leads to an improved peeling effect compared to conventional peeling compositions.

In particular, the substrate-based product allows the reduction of hyperpigmentation and/or hypopigmentation of the skin, more particularly allows the reduction of hyperpigmentation such as erythema and/or redness of the skin. The substrate-based product according to the invention is thus useful for reducing the aspect of spots, freckles, frecks or chloasma.

In another embodiment, the substrate-based product of the invention increases the lightness of the skin. The substrate-based product according to the invention thus may allow a more homogenous skin complexion and/or lighten the skin complexion.

In another embodiment, the substrate-based product is useful as an anti-ageing product, in particular by reducing the fine lines and wrinkles of the skin.

Moreover, the substrate-based product is convenient to use by the consumers in a single step, without dripping off from the application zone of the skin. The substrate-based product of the invention is easy to apply and to remove from the zone of skin to be peeled, compared to conventional peeling compositions.

Advantageously, the consumers may also use a kit comprising the substrate-based product and an aqueous composition.

The substrate-based product according to the invention has also a good stability at low pH. In one embodiment, the pH of the substrate-based product is comprised between 0.1 and 5.5.

In one embodiment, the term “substrate-based product” refers to the substrate-based product and to the activated substrate-based product, as defined below.

### Substrate-Based Product

In one embodiment, the substrate-based product is a substrate-based cosmetic product.

In a particular embodiment, the substrate-based product is a substrate-based cosmetic mask. In one embodiment, the substrate-based cosmetic mask of the invention is a skin mask, being intended as a body or a face mask, preferably a face mask.

As used herein term “face mask” may refer to a sheet in the shape of face and generally comprising designed areas, for example openings for eyes, and/or nose and/or mouth. Such areas are generally cut in the sheet. The mask may also comprise a slit delimiting a flapper intended to be pushed away by the nose of the user, to delimit a nose insertion through opening.

When used as a body mask, the sheet may have any desired shape, such as a rectangular or square shape, of any dimensions. In one embodiment, the substrate-based product presents a shape corresponding to the skin zone on which said substrate-based product has to be placed. In a particular embodiment, the skin zone is the skin located on a member of the user such as an arm or a leg.

In another particular embodiment, said substrate-based product is solid. In one embodiment, the substrate-based product retains its shape and does not disintegrate and/or dissolve during the application time on the skin.

### Water-Insoluble Substrate

By “water-insoluble substrate”, it may be meant a substrate which does not dissolve (i.e. does not form a solution) or readily break apart upon immersion in water, for example a substrate which does not dissolve after one day in water at room temperature, which ranges between 20° C. and 25° C., and atmospheric pressure (about 1 013.25 hPa).

In one embodiment, the water-insoluble substrate is a sheet, preferably a dry sheet.

In one embodiment, the water-insoluble substrate comprises at least one fibrous layer. The water-insoluble substrate may have a structure of woven, knitted, non-woven and/or polymeric mesh. In a particular embodiment, the water-insoluble substrate is made of a non-woven product, such as a non-woven sheet.

By “non-woven”, it may be meant, a product including fibers in which the individual fibers or filaments are arranged in a disordered manner in a sheet-like structure and which are neither woven nor knit. The fibers of the non-woven product are generally linked to one another, under the effect of a mechanical action for example, by needle-punching, air jet or water jet, or under the effect of a thermal action, or by adding a binder. Such a non-woven product is, for example, defined by standard ISO 9092, as a web or a sheet of fibers oriented directionally or at random, bound by friction and/or cohesion and/or adhesion, in particular excluding paper or products obtained by weaving, knitting, tufting or stitching incorporating threads or bonding filaments.

The difference between a non-woven product and a paper may be the absence of hydrogen bonds between the fibers in a non-woven product.

In one embodiment, the fibers are chosen among the group consisting of: natural fibers, synthetic fibers and mixtures thereof. Among synthetic fibers it can be cited polyester; polyolefin such as polypropylene or polyethylene; polyamide such as nylon 6 or nylon 66; viscose; acrylic fibers; modacrylic fibers; polyvinylidene chloride and Spandex (also called elastane). Among natural fibers it can be cited cellulosic fibers, such as wood pulp, cotton, hemp, jute, and flax fibers; silk; and keratin, such as wool and camel hair fibers.

In one embodiment, the water-insoluble substrate typically has a density of 30 g/m² to 400 g/m², preferably 40 g/m² to 80 g/m².

In a particular embodiment, the water-insoluble substrate is non-woven viscose.

### Skin Binding Agent

By “skin binding agent”, it may be understood a polymer which has adhesive properties in liquid stage and which hardens upon the drying.

In one embodiment, the skin binding agent is a water soluble polymer. In another embodiment, the skin binding agent is a water soluble filmogenic polymer or a thickener.

A filmogenic polymer is adapted to form alone or with other filmogenic agents, a continuous film, preferably adhering to skin.

By “water soluble polymer” is meant a polymer which upon immersion in water at 25° C. disintegrate and/or dissolve slowly e.g. over period of several minutes to several days without stirring.

By “thickener” is meant a substance which increases the viscosity of a composition without substantially modifying its other properties.

In one embodiment, the skin binding agent is selected among natural and synthetic adhesive polymers.

In one embodiment, the skin binding agent is selected from the group consisting of:

vegetable starches, casein, gelatin, polyacrylamides, polyvinylalcohol, polyacrylic acid, polyamines, polyethyleneimines, polyvinylpyrrolidone copolymers (PVP), polyethylene glycols, cellulose and its derivatives such as methylcellulose derivatives, quaternary ammonium cellulose, carboxymethyl cellulose, xanthan gum, agar, pectin gum, guar gum and their derivatives, carboxypolymethylene, and their mixtures.

Among the polyacrylamides, it can be cited as non-limiting examples sodium polyacrylates, potassium polyacrylates, crosslinked polyacrylamides, and commercially available grades of such polymers e.g. Hydrosorb), Soilsorb®.

Non-limiting examples of polyvinylalcohols are PVA 4-88, PVA 5-88, PVA 8-88, PVA 18-88, PVA 26-88, PVA 40-88, BE-26, BE-24E and BE-28 (classified on the basis of viscosity and level of hydrolysis or Molecular weight).

Among the polyvinylpyrrolidone copolymers, it can be cited as non-limiting examples PVP K-15, PVP K-30, PVP K-60, PVP K-90 and PVP K-120 (classified on the basis of viscosity and level of hydrolysis or Molecular weight).

Non limiting examples of polyethylene glycols are PEG-200, PEG-300, PEG-400, PEG-600, PEG-1000, PEG-1500, PEG-2000, PEG-4000 and PEG-6000.

### Chemical Exfoliator

By “chemical exfoliator” it may be understood substances which have ability to remove dead skin cells by breaking the bond between cells.

In one embodiment, the chemical exfoliator is selected from the group consisting of:

hydroxyacids, ascorbic acid and its derivatives, allantoin glycolate, allantoin mandelate, allantoin lactate, allantoin citrate, glucosamine mandelate, glucosamine ascorbate, glucosamine citrate, glucosamine phytate, glucosamine salicylate, glucosamine hydronate, creatinine lactate, creatinine glycolate, creatinine hydroxy citrate, creatinine citrate, niacinamide glycolate, niacinamide citrate, niacinamide salicylate, pyridoxine lactate, pyridoxine glycolate, pyridoxine malate, pyridoxine salicylate, chitosan lactate, chitosan citrate, chitosan malate, and their mixtures.

In a particular embodiment, the chemical exfoliator is chosen among the alpha-hydroxy-acids and/or the beta-hydroxy-acids.

Alpha-hydroxy acids, or α-hydroxy acids (AHAs), are a class of organic compounds that comprise a carboxylic acid substituted with a hydroxyl group on the adjacent carbon. Among alpha-hydroxy-acids, it can be cited glycolic acid, lactic acid, malic acid, mandelic acid, tartaric acid and citric acid.

A beta hydroxy acid or 3-hydroxy acid (BHA) is an organic compound that contains a carboxylic acid functional group and hydroxyl group separated by two carbon atoms. As an example of beta-hydroxy-acids, salicylic acid may be mentioned.

In another particular embodiment, the chemical exfoliator is chosen among the group consisting of:

ascorbic acid, glycolic acid, lactic acid, malic acid, mandelic acid, tartaric acid, citric acid, salicylic acid, and their mixtures.

In one embodiment, the chemical exfoliator is chosen among the group consisting of: mandelic acid, salicylic acid, ascorbic acid, and their mixtures, preferably ascorbic acid.

In one embodiment, the substrate-based product according to the invention further comprises at least one skin care agent. In one embodiment, the skin care agent is selected from the group consisting of:

humectants or moisturizers, whitening agents, anti-oxidants, cleansing agents, free radical scavengers, skin tone altering agents, anti-acne agents, anti-pimple agents, anti-aging agents, anti-wrinkle agents, anti-inflammatory agents, soothing agents, skin texture treatment agents, anti-perspirant agents, anti-bacterial agents, nourishing agents, sebum and/or moisture absorbers, and any combination thereof.

In one embodiment, the substrate-based product according to the invention further comprises at least one physiologically acceptable excipient. In one embodiment, the physiologically acceptable excipient is selected from the group consisting of:

oils, surfactants, structuring agents, sunscreen filters, preservatives, acidifying and/or alkalizing agents, buffering agents, chelating agents, coloring additives, and any combination thereof.

In a particular embodiment, the substrate-based product comprises:
a water-insoluble substrate such as viscose,
ascorbic acid,
polyvinylpyrrolidone, and
an antioxidant, such as ferulic acid.

In an another embodiment, the substrate-based product comprises:
a water-insoluble substrate such as viscose,
polyvinylpyrrolidone,
mandelic acid and salicylic acid, and
an antioxidant, such as ferulic acid.

### Preparation Composition

The preparation composition comprises at least one chemical exfoliator and at least one skin binding agent as defined above. In a particular embodiment, the chemical exfoliator is ascorbic acid and the skin binding agent is polyvinylpyrrolidone.

In one embodiment, the preparation composition further comprises at least one skin care agent as defined above.

In one embodiment, the preparation composition further comprises at least one physiologically acceptable excipient as defined above.

In a particular embodiment, the skin binding agent is comprised between 0.1% and 99.99% by weight, preferably between 1% and 50% by weight, even more preferably between 5% and 30% by weight, in respect of the total weight of the preparation composition.

In another particular embodiment, the chemical exfoliator in the preparation composition is comprised between 0.1% and 99.99% by weight, preferably between 1% and 50% by weight, more preferably between 2% and 30% by weight, in respect of the total weight of the preparation composition.

In one embodiment, the amount of a skin care active agent is comprised between 0.001% and 99% by weight, more preferably between 0.01% and 30% by weight, even more preferably between 0.01% and 5% by weight, in respect of the total weight of the preparation composition.

In one embodiment, the preparation composition as defined above comprises at least 50% by weight of water, in respect of the total weight of the preparation composition.

In one embodiment, the pH of the preparation composition is in acidic range, for example it ranges from 1 to 6, and preferably from 2 to 5.

The invention further relates to the use of the preparation composition as defined above for the preparation of the substrate-based product as defined herein.

### Method of Preparation of the Substrate-Based Product

The invention relates to a method for preparing a substrate-based product as defined above, comprising a step of application of the preparation composition defined above on a water-insoluble substrate.

In one embodiment, the preparation composition as defined above is applied on the water-insoluble substrate using any known coating technology such as padding mangle, screen printing, digital printing, knife coating or spray coating.

The density of the wet substrate may depend on the nature of chemical exfoliator and skin binder used and may be comprised between 100 g/m² and 700 g/m², advantageously from 200 g/m² to 600 g/m², and more preferably from 300 g/m² to 600 g/m².

In one embodiment, said method of preparation further comprises a step of drying the water-insoluble substrate.

In one embodiment, drying of the wet water-insoluble substrate is done by known techniques of the skilled person in the art. Natural drying is obtained by just waiting without using any specific mechanical means or temperature increase.

Drying may be performed manually, semi-automatically or by an automatic process. The drying temperature may depend upon thermal properties of the water-insoluble substrate and the preparation composition. Drying may enable eliminating water from the water-insoluble substrate, partly or completely. In one embodiment, the temperature of the drying step varies from 25° C. to 100° C., more preferably 35° C. to 70° C. and even more preferably from 40° C. to 60° C. In one embodiment, the drying is done in presence of vacuum.

### Applications

The invention relates to a method of cosmetic care comprising the following steps:
a) wetting with a physiologically acceptable aqueous composition at least one zone of the substrate-based product as defined above,
b) applying the activated substrate-based product on at least one zone of skin,
c) drying the activated substrate-based product, and
d) removing the activated substrate-based product from the skin.

The aqueous composition can be a solution, a suspension or an oil-in-water emulsion.

The term emulsion may refer to a macroscopically homogeneous but microscopically heterogeneous mixture of two non-miscible phases.

In one embodiment, the oil-in-water emulsion comprises:
an oily phase, optionally comprising lipophilic skin care actives such as vitamin E and its derivatives, vitamin A and its derivatives, or their mixtures,
an aqueous phase such as water, and
at least one surfactant with a HLB (hydrophilic-lipophilic balance) value in the range from 8 to 12, or a combination of at least two surfactants to achieve a HLB value of the combination in the range of 8 to 12.

In one embodiment, the surfactant(s) is(are) selected from the group consisting of: glyceryl stearate, glyceryl stearate self-emulsifying, PEG-100 stearate, steareth-2 and steareth-20 combination, methylglucose sesquistearate and its polyglyceryl derivatives, laureth/oleath derivatives, PEG-8 dioleate, sorbitan laurate and other sorbitan esters, PEG-7 glyceryl cocoate, PEG-20 almond glycerides, and their mixtures.

In one embodiment, the aqueous composition is water. In one embodiment, the aqueous composition comprises at least 60% by weight of water, in relation to the total weight of the composition. In one embodiment, said physiologically acceptable aqueous composition further comprises at least one skin care agent as previously defined, preferably a moisturizing agent.

In one embodiment, the physiologically acceptable aqueous composition is applied on the whole surface of the substrate-based product. In one embodiment, the substrate-based product is wet by spraying the aqueous composition on said substrate-based product, or by dipping said substrate-based product into the aqueous composition. The quantity of the aqueous composition to be applied on the substrate-based product depends on the type of the water-insoluble substrate and the preparation composition used for its preparation. It may be sufficient to provide a good adhesion on the skin of the substrate-based product.

By “activated substrate-base product” is meant the wet substrate-based product, in particular thanks to the aqueous composition defined above.

In one embodiment, the drying is performed naturally.

In one embodiment, after applying the activated substrate-based product on at least one skin zone, said method comprises a step of waiting a sufficient period of time, called “application time”, until said activated substrate-based product has naturally dried, and then removing said dried activated substrate-based product by peeling it off from said skin zone.

After the application time, said dried activated substrate-based product can be easily peeled off from skin.

In one embodiment, the application time is comprised between 10 and 60 minutes, more preferably between 15 and 30 minutes, yet more preferably between 15 and 20 minutes.

In one embodiment, said method of cosmetic care is a skin smoothening cosmetic treatment.

In one embodiment, said method of cosmetic care is an anti-ageing cosmetic treatment.

In a particular embodiment, said method of cosmetic care is a non-therapeutic cosmetic treatment of skin conditions such as acne, scars, wound scars, fine lines, wrinkles, erythema, and other dermal tissue disfigurements, preferably erythema.

In another particular embodiment, said method of cosmetic care is a non-therapeutic cosmetic treatment of skin conditions such as pimples, scars, wound scars, fine lines, wrinkles, erythema, and other dermal tissue disfigurements.

The invention also relates to a method of treatment comprising the following steps:
a) wetting with a physiologically acceptable aqueous composition at least one zone of the substrate-based product as defined above,
b) applying the activated substrate-based product on at least one zone of skin, and
c) drying the activated substrate-based product, and
d) removing the activated substrate-based product from the skin.

In a particular embodiment, said method is a method of treatment of skin conditions such as acne, scars, wound scars, fine lines, wrinkles, erythema, and other dermal tissue disfigurements.

The invention also relates to a kit comprising the substrate-based product as above, and a physiologically acceptable aqueous composition as defined above, optionally comprising at least one skin care agent as defined above. In one embodiment, the kit comprises a plurality of substrate-based products and a physiologically acceptable aqueous composition.

In one embodiment, the physiologically acceptable aqueous composition is contained in a spray bottle.

In one embodiment, the activated substrate-base product and/or the kit is(are) intended for a one time use.

### DESCRIPTION OF THE FIGURES

FIG. 1 represents the percentage of evolution of the “a” value, considered as a representation of erythema, in function of the time after removal from the skin.

FIG. 2 represents the percentage of evolution of the lightness of the skin “L”, in function of the time after removal from the skin.

“T-imm” refers to the time at which the substrate-based product was removed from the skin, after 15 minutes of application.

Throughout the application, the term “comprising a” or “including a” means “comprising at least one” or “including at least one”, unless otherwise specified.

Throughout the above description, unless specified otherwise, the term “between x and y” refers to an inclusive range, i.e. the values x and y are included in the range.

The invention will now be illustrated in the following non-limiting examples. Unless specified otherwise, the % are expressed by weight in relation to the total weight of the composition.

### EXAMPLES

### Example 1

1. Preparation of a Substrate-Based Product According to the Invention:

A preparation composition was prepared as follows:

| Ingredient | % by weight |
|---|---|
| Ascorbic Acid | 15 |
| Polyvinylpyrrolidone | 30 |
| Ferulic Acid | 0.5 |
| Water | 54.5 |
| Total | 100 |

The preparation composition was coated using a knife on roll doctor blade coating system, well-known by the skilled person in the art, on non-woven viscose and dried at 60 C.° by hot air drying technique.

2. Comparative Test:

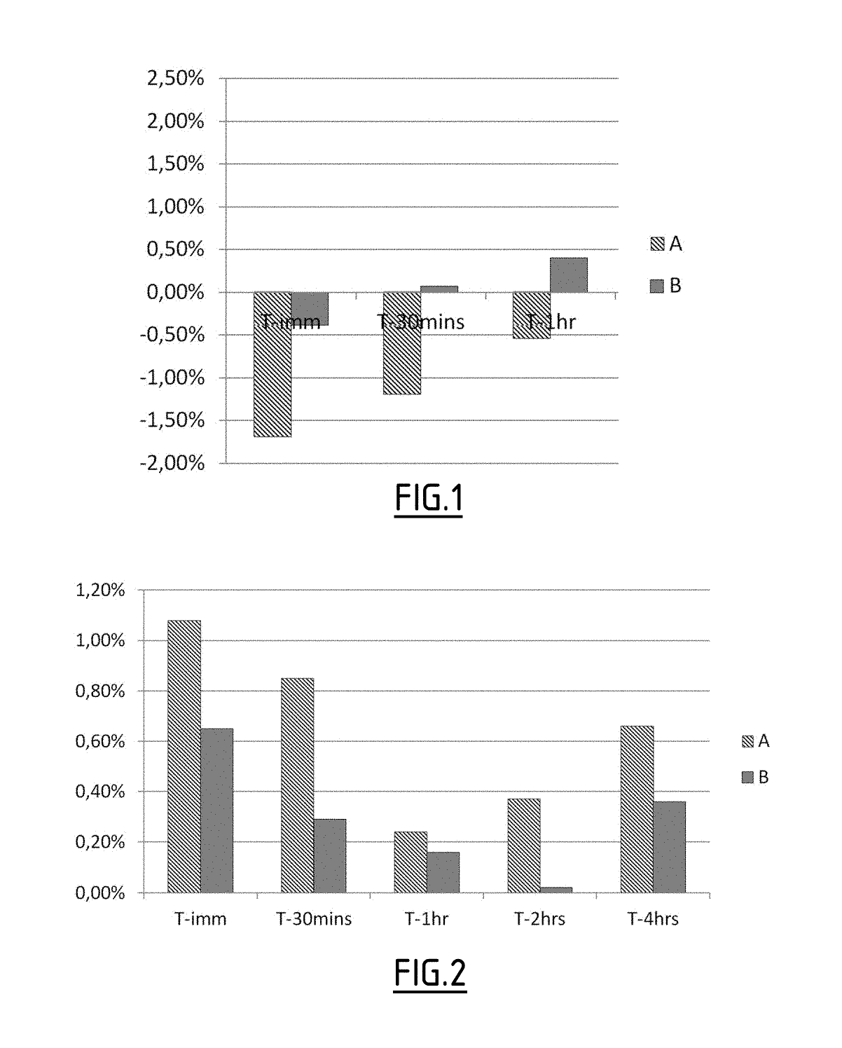

The substrate-based product obtained as described above was cut into the size of 2×2 inches patches and wetted with 0.5 gram of water in order to obtain an activated substrate-based product (mentioned as Product A in FIGS. 1 and 2).

These activated substrate-based products were applied on forearm of 16 users for 15 minutes. After 15 minutes, patches were removed and color measurements were taken using a spectrometer (model n^{o} CM 2600D from Konica Minolta).

Corresponding preparation compositions (mentioned as Product B in FIGS. 1 and 2) were also applied directly on forearm of users, on 2×2 inches area for 15 minutes and were after rinsed off with water.

The results are shown in FIGS. 1 and 2.

Both FIGS. 1 and 2 represent a percentage of evolution, respectively of value a and L. “a” value represents the redness of the skin and “L” the lightness of the skin, measured directly by the spectrometer.

The percentage of evolution is the average of measurements of the treated area (product) minus the average of measurements for non-treated area (bare skin), calculated as follows:

$$Percentage Evolution = \frac{( {T}_{n - product} - {T}_{0 - product} )}{{T}_{0 - product}} - \frac{( {T}_{n - bareskin} - {T}_{0 - bareskin} )}{{T}_{n - bareskin}} \times 100 \%$$

FIG. 1 shows that product A gives a higher negative “a” percentage of evolution than product B, meaning that when the substrate-based product according to the invention is used, it shows higher efficacy than the preparation composition alone, in terms of reducing redness of the skin.

FIG. 2 shows that product A gives a higher “L” percentage of evolution than product B meaning that when the substrate-based product according to the invention is used, it shows higher efficacy than the preparation composition alone, in terms of lightening of the skin, even 4 hours after application.

FIGS. 1 and 2 show that use of the substrate-based product according to the invention reduce the value of erythema, the redness of the skin and enhances the lightness value.

1. A substrate-based product comprising:
a water-insoluble substrate,
at least one chemical exfoliator, and
at least one skin binding agent.

2. The substrate-based product according to claim 1, said substrate-based product being a cosmetic mask, preferably a cosmetic skin mask.

3. The substrate-based product according to claim 2, said substrate-based product being a cosmetic face mask.

4. The substrate-based product according to claim 1, wherein the skin binding agent is a water soluble polymer.

5. The substrate-based product according to claim 1, wherein the skin binding agent is selected from the group consisting of:
vegetable starches, casein, gelatin, polyacrylamides, polyvinylalcohol, polyacrylic acid, polyamines, polyethyleneimines, polyvinylpyrrolidone copolymers (PVP), polyethylene glycols, cellulose and its derivatives, xanthan gum, agar gum, pectin gum, guar gum and their derivatives, carboxypolymethylene, and their mixtures.

6. The substrate-based product according to claim 1, wherein the chemical exfoliator is selected from the group consisting of:
hydroxyacids, ascorbic acid and its derivatives, allantoin glycolate, allantoin mandelate, allantoin lactate, allantoin citrate, glucosamine mandelate, glucosamine ascorbate, glucosamine citrate, glucosamine phytate, glucosamine salicylate, glucosamine hydronate, creatinine lactate, creatinine glycolate, creatinine hydroxycitrate, creatinine citrate, niacinamide glycolate, niacinamide citrate, niacinamide salicylate, pyridoxine lactate, pyridoxine glycolate, pyridoxine malate, pyridoxine salicylate, chitosan lactate, chitosan citrate, chitosan malate, and their mixtures.

7. The substrate-based product according to claim 1, wherein the chemical exfoliator is selected from the group consisting of:
ascorbic acid, glycolic acid, lactic acid, malic acid, mandelic acid, tartaric acid, citric acid, salicylic acid, and their mixtures.

8. The substrate-based product according to claim 1, further comprising at least one skin care agent.

9. The substrate-based product according to claim 1, further comprising at least one physiologically acceptable excipient.

10. A method for preparing a substrate-based product as defined in claim 1, comprising a step of application of a preparation composition comprising at least one chemical exfoliator and at least one skin binding agent on a water-insoluble substrate.

11. A method of cosmetic care comprising the following steps:
a) wetting with a physiologically acceptable aqueous composition at least one zone of the substrate-based product according to claim 1,
b) applying the activated substrate-based product on at least one zone of skin,
c) drying the activated substrate-based product, and
d) removing the activated substrate-based product from the skin.

12. A kit comprising the substrate-based product as defined in claim 1, and a physiologically acceptable aqueous composition, optionally comprising at least one skin care agent.

13. The substrate-based product according to claim 2, wherein the skin binding agent is a water soluble polymer.

14. The substrate-based product according to claim 3, wherein the skin binding agent is a water soluble polymer.

15. The substrate-based product according to claim 2, wherein the skin binding agent is selected from the group consisting of: vegetable starches, casein, gelatin, polyacrylamides, polyvinylalcohol, polyacrylic acid, polyamines, polyethyleneimines, polyvinylpyrrolidone copolymers (PVP), polyethylene glycols, cellulose and its derivatives, xanthan gum, agar gum, pectin gum, guar gum and their derivatives, carboxypolymethylene, and their mixtures.

16. The substrate-based product according to claim 3, wherein the skin binding agent is selected from the group consisting of: vegetable starches, casein, gelatin, polyacrylamides, polyvinylalcohol, polyacrylic acid, polyamines, polyethyleneimines, polyvinylpyrrolidone copolymers (PVP), polyethylene glycols, cellulose and its derivatives, xanthan gum, agar gum, pectin gum, guar gum and their derivatives, carboxypolymethylene, and their mixtures.

17. The substrate-based product according to claim 4, wherein the skin binding agent is selected from the group consisting of: vegetable starches, casein, gelatin, polyacrylamides, polyvinylalcohol, polyacrylic acid, polyamines, polyethyleneimines, polyvinylpyrrolidone copolymers (PVP), polyethylene glycols, cellulose and its derivatives, xanthan gum, agar gum, pectin gum, guar gum and their derivatives, carboxypolymethylene, and their mixtures.

18. The substrate-based product according to claim 2, wherein the chemical exfoliator is selected from the group consisting of: hydroxyacids, ascorbic acid and its derivatives, allantoin glycolate, allantoin mandelate, allantoin lactate, allantoin citrate, glucosamine mandelate, glucosamine ascorbate, glucosamine citrate, glucosamine phytate, glucosamine salicylate, glucosamine hydronate, creatinine lactate, creatinine glycolate, creatinine hydroxycitrate, creatinine citrate, niacinamide glycolate, niacinamide citrate, niacinamide salicylate, pyridoxine lactate, pyridoxine glycolate, pyridoxine malate, pyridoxine salicylate, chitosan lactate, chitosan citrate, chitosan malate, and their mixtures.

19. The substrate-based product according to claim 3, wherein the chemical exfoliator is selected from the group consisting of: hydroxyacids, ascorbic acid and its derivatives, allantoin glycolate, allantoin mandelate, allantoin lactate, allantoin citrate, glucosamine mandelate, glucosamine ascorbate, glucosamine citrate, glucosamine phytate, glucosamine salicylate, glucosamine hydronate, creatinine lactate, creatinine glycolate, creatinine hydroxycitrate, creatinine citrate, niacinamide glycolate, niacinamide citrate, niacinamide salicylate, pyridoxine lactate, pyridoxine glycolate, pyridoxine malate, pyridoxine salicylate, chitosan lactate, chitosan citrate, chitosan malate, and their mixtures.

20. The substrate-based product according to claim 4, wherein the chemical exfoliator is selected from the group consisting of hydroxyacids, ascorbic acid and its derivatives, allantoin glycolate, allantoin mandelate, allantoin lactate, allantoin citrate, glucosamine mandelate, glucosamine ascorbate, glucosamine citrate, glucosamine phytate, glucosamine salicylate, glucosamine hydronate, creatinine lactate, creatinine glycolate, creatinine hydroxycitrate, creatinine citrate, niacinamide glycolate, niacinamide citrate, niacinamide salicylate, pyridoxine lactate, pyridoxine glycolate, pyridoxine malate, pyridoxine salicylate, chitosan lactate, chitosan citrate, chitosan malate, and their mixtures.
